SEQUENCE LISTING (2 sequences)

SEQ ID NO: 1 - DNA, 678 bp, Clostridium difficile
ttgaaaaaattatatagaatagtaattaacattatactagtattagtaatactttacagtggatttaatatatactcaaaacttaccaaatacaatcatgatactaaaatatcctcagaacttcaaaaaaaagaatataaaaaagaggatttatcaaagattaacagtgattttaaattttggctttctgttgagaatacaaatataaattatccagttgtacaatctaaagataattcttactatcttgacaaagatttttataaaaaagattctatttctggtacactgtttatggattatagaaataaatctattgatgacaaaaatatcataatatatggacataatatgaaaaataaaactatgttcaacaacctaaataagtttaaagatgctgacttctttaaaaaaaataataaaataaaaataaccttgaatggaaaagaatttttatatgatgttttttctgcttatattgtagaatctgattatgattatctaaaaacaaattttaataatgaatctgattatcaaaattacataaatgatattacttctaaatcattgtataaatctcctattaaggtaaatagcaatgataaaatagttacactttctacatgtacctatgaatttgatgatgccagaatggtgattcatggtagattgatttaa

SEQ ID NO: 2 - protein, 225 aa, Clostridium difficile
LKKLYRIVINIILVLVILYSGFNIYSKLTKYNHDTKISSELQKKEYKKEDLSKINSDFKFWLSVENTNINYPVVQSKDNSYYLDKDFYKKDSISGTLFMDYRNKSIDDKNIIIYGHNMKNKTMFNNLNKFKDADFFKKNNKIKITLNGKEFLYDVFSAYIVESDYDYLKTNFNNESDYQNYINDITSKSLYKSPIKVNSNDKIVTLSTCTYEFDDARMVIHGRLI

(19) United States
(12) Patent Application Publication
(10) Pub. No.: US 2012/0301428 A1
(43) Pub. Date: Nov. 29, 2012

(54) CLOSTRIDIUM GENE

(76) Inventors: Brendan Wren, London (GB); Lisa Dawson, London (GB)

(21) Appl. No.: 13/575,733

(22) PCT Filed: Feb. 22, 2011
(86) PCT No.: PCT/GB2011/050337

§ 371 Date: Jul. 27, 2012

(30) Foreign Application Priority Data
Feb. 24, 2010 (GB) 1003089.8

(51) Int. Cl. A61K 39/08; C12N 9/52; A61P 31/04; A61K 38/20; A61K 38/21; C12Q 1/37; C12N 15/57

(52) U.S. Cl. 424/85.2; 435/23; 435/220; 536/23.2; 424/190.1; 424/85.5; 424/85.7; 424/85.6

(57) ABSTRACT

The disclosure relates to the identification of an essential Clostridium difficile gene that encodes a polypeptide with protease activity and its use in the identification of anti-microbial agents and as antigen in subunit vaccines.

The invention relates to the identification of an essential Clostridium difficile gene that encodes a polypeptide with protease activity and its use in the identification of anti-microbial agents and as antigen in subunit vaccines.

Clostridium is a genus of gram-positive bacteria which are obligate anaerobes some of which are significant human pathogens. For example, C. difficile is a major cause of infection in hospitals with conditions varying from mild antibiotic-associated diarrhoea/colitis to life threatening conditions such as pseudomembranous colitis. The disease is manifest through the administration of broad-spectrum antibiotics which deplete the gut microflora allowing C. difficile to proliferate and cause disease mediated through toxins. Treatment usually involves antibiotic therapy, i.e. vancomycin or metronidazole, but these can exacerbate the disease. There is a clear need to develop agents that will specifically kill or disable C. difficile without disturbing the natural microflora of the gut, or for subunit vaccines that would confer protection to vulnerable patients. C. difficile forms spores that are resistant to heat, radiation, chemical disinfectants and dessication. Moreover, the spores are resistant to antibiotic treatment making C. difficile a very recalcitrant microbial pathogen. Further examples of Clostridium species that cause human disease are C. botulinum, which produces a toxin that causes botulism; C. perfringens, which causes a number of conditions, which include food poisoning and gangrene; and C. tetani which causes tetanus.

There is a clear desire and need to identify agents that can control Clostridium infection and this is assisted by the identification of genes that encode proteins that are essential for the survival of the microbe and/or spore. This can either be via identification of small molecule inhibitors that antagonize the activity of essential proteins, the development of vaccines that target and inactivate the essential protein; or the development of therapeutic monoclonal antibodies that bind and inactivate the essential protein.

Vaccines protect against a wide variety of infectious diseases. Many modern vaccines are therefore made from protective antigens of the pathogen, which are isolated by molecular cloning and purified. These vaccines are known as ‘subunit vaccines’. The development of subunit vaccines has been the focus of considerable research in recent years. The emergence of new pathogens and the growth of antibiotic resistance have created a need to develop new vaccines and to identify further candidate molecules useful in the development of subunit vaccines. Likewise the discovery of novel vaccine antigens from genomic and proteomic studies is enabling the development of new subunit vaccine candidates, particularly against bacterial pathogens. However, although subunit vaccines tend to avoid the side effects of killed or attenuated pathogen vaccines, their ‘pure’ status means that subunit vaccines do not always have adequate immunogenicity to confer protection.

The Sortase B (SrtB) or subfamily-2 sortases are membrane cysteine transpeptidases found in gram-positive bacteria that anchor surface proteins to peptidoglycans of the bacterial cell wall envelope. This involves a transpeptidation reaction in which the surface protein substrate is cleaved at a conserved cell wall sorting signal and covalently linked to peptidoglycan for display on the bacterial surface. Sortases are grouped into different classes and subfamilies based on sequence, membrane topology, genomic positioning, and cleavage site preference. Sortase B cleaves surface protein precursors between threonine and asparagine at a conserved NPQTN motif with subsequent covalent linkage to peptidoglycan. It is required for anchoring the heme-iron binding surface protein IsdC to the cell wall envelope and the gene encoding Sortase B is located within the isd locus in S. aureus and B. anthracis. It may also play a role in pathogenesis. Sortase B contains an N-terminal region that functions as both a signal peptide for secretion and a stop-transfer signal for membrane anchoring. At the C-terminus, it contains the catalytic TLXTC signature sequence, where X is usually a serine. Genes encoding SrtB and its targets are generally clustered in the same locus.

This disclosure relates to the characterization of a C. difficile Sortase B gene, CD2718 in strain 630, and the discovery that it is an essential gene for the viability of the C. difficile cell.

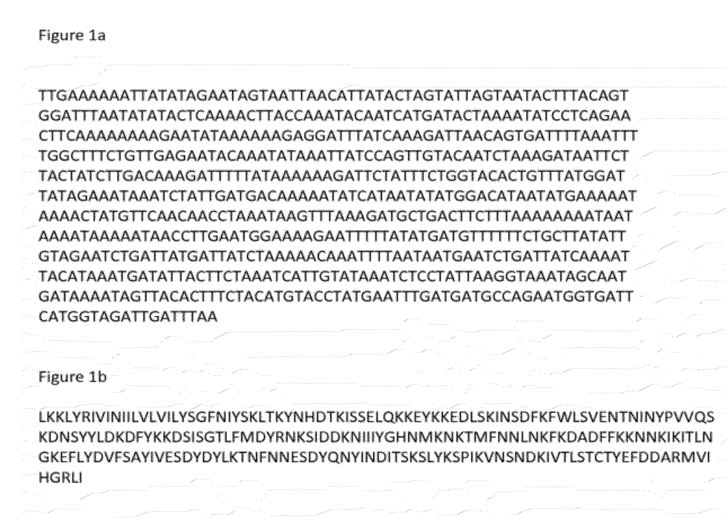

According to an aspect of the invention there is provided the use of a polypeptide encoded by a nucleic acid molecule comprising a nucleotide sequence as represented in FIG. 1a, or a nucleic acid molecule that hybridizes under stringent hybridization conditions to a nucleotide sequence comprising FIG. 1a, and which encodes a polypeptide with protease activity, for the identification of agents that modulate the activity of said polypeptide.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×−6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×−3×SSC at RT to 55° C. for 20-30 minutes each.

According to an aspect of the invention there is provided a screening method for the identification of an agent that has protease inhibitory activity comprising the steps of:
i) providing a polypeptide encoded by a nucleic acid molecule selected from the group consisting of:
   a) a nucleic acid molecule comprising a nucleotide sequence as represented in FIG. 1a;
   b) a nucleic acid molecule comprising nucleotide sequences that hybridise to the sequence identified in (a) above under stringent hybridization conditions and which encodes a polypeptide that has protease activity;
ii) providing at least one candidate agent to be tested;
iii) forming a preparation that is a combination of (i) and (ii) above; and
iv) testing the effect of said agent on the activity of said polypeptide.

In a further preferred method of the invention said polypeptide comprises or consists of the amino acid sequence in FIG. 1b, or active part thereof.

According to a further aspect of the invention there is provided a modelling method to determine the association of an agent with a protease polypeptide comprising the steps of:
i) providing computational means to perform a fitting operation between an agent and a polypeptide comprising or consisting of the amino acid sequence in FIG. 1b; and
ii) analysing the results of said fitting operation to quantify the association between the agent and the polypeptide.

The rational design of binding entities for proteins is known in the art and there are a large number of computer programs that can be utilised in the modelling of 3-dimensional protein structures to determine the binding of chemical entities to functional regions of proteins and also to determine the effects of mutation on protein structure. This may be applied to binding entities and also to the binding sites for such entities. The computational design of proteins and/or protein ligands demands various computational analyses which are necessary to determine whether a molecule is sufficiently similar to the target protein or polypeptide. Such analyses may be carried out in current software applications, such as the Molecular Similarity application of QUANTA (Molecular Simulations Inc., Waltham, Mass.) version 3.3, and as described in the accompanying User's Guide, Volume 3 pages. 134-135. The Molecular Similarity application permits comparisons between different structures, different conformations of the same structure, and different parts of the same structure. Each structure is identified by a name. One structure is identified as the target (i.e., the fixed structure); all remaining structures are working structures (i.e. moving structures). When a rigid fitting method is used, the working structure is translated and rotated to obtain an optimum fit with the target structure.

The person skilled in the art may use one of several methods to screen chemical entities or fragments for their ability to associate with a target. The screening process may begin by visual inspection of the target on the computer screen, generated from a machine-readable storage medium. Selected fragments or chemical entities may then be positioned in a variety of orientations, or docked, within the binding pocket.

Useful programs to aid the person skilled in the art in connecting the individual chemical entities or fragments include: CAVEAT (P. A. Bartlett et al, “CAVEAT: A Program to Facilitate the Structure-Derived Design of Biologically Active Molecules”. In Molecular Recognition in Chemical and Biological Problems”, Special Pub., Royal Chem. Soc., 78, pp. 182-196 (1989)). CAVEAT is available from the University of California, Berkeley, Calif. 3D Database systems such as MACCS-3D (MDL Information Systems, San Leandro, Calif.). This is reviewed in Y. C. Martin, “3D Database Searching in Drug Design”, J. Med. Chem., 35, pp. 2145-2154 (1992); and HOOK (available from Molecular Simulations, Burlington, Mass.).

Once the agent has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. The computational analysis and design of molecules, as well as software and computer systems are described in U.S. Pat. No. 5,978,740 which is included herein by reference.

According to an aspect of the invention there is provided a polypeptide selected from the group consisting of:
i) a polypeptide encoded by a nucleotide sequence as represented in FIG. 1a, or an antigenic fragment thereof;
ii) a polypeptide encoded by a nucleotide sequence wherein said sequence is degenerate as a result of the genetic code to the nucleotide sequence defined in (i) and which has protease activity;
iii) a polypeptide comprising an amino acid sequence wherein said sequence is modified by addition deletion or substitution of at least one amino acid residue as represented in FIG. 1b, wherein said polypeptide is for use as a vaccine.

A modified polypeptide or variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In one embodiment, the variant polypeptides have at least 85% identity, more preferably at least 90% identity, even more preferably at least 95% identity, still more preferably at least 97% identity, and most preferably at least 99% identity with the full length amino acid sequences illustrated herein.

In a preferred embodiment of the invention said polypeptide is encoded by a nucleotide sequence as represented in FIG. 1a.

In an alternative preferred embodiment of the invention said polypeptide is represented by the amino acid sequence in FIG. 1b, or antigenic part thereof.

According to a further aspect of the invention there is provided a nucleic acid molecule that encodes a polypeptide according to the invention for use as a vaccine.

According to a further aspect of the invention there is provided a vaccine composition for use in the vaccination against a microbial infection, comprising a polypeptide selected from the group consisting of:
i) a polypeptide encoded by a nucleotide sequence as represented in FIG. 1a, or an antigenic fragment thereof;
ii) a polypeptide encoded by a nucleotide sequence wherein said sequence is degenerate as a result of the genetic code to the nucleotide sequence defined in (i);
iii) a polypeptide comprising an amino acid sequence wherein said sequence is modified by addition deletion or substitution of at least one amino acid residue as represented in FIG. 1b and which retains protease activity; wherein said composition optionally includes an adjuvant and/or carrier.

In a preferred embodiment of the invention said composition includes an adjuvant and/or carrier.

In a preferred embodiment of the invention said adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

In a further alternative embodiment of the invention said adjuvant is a TLR agonist such as CpG oligonucleotides, flagellin, monophosphoryl lipid A, poly I:C and derivatives thereof.

In a preferred embodiment of the invention said adjuvant is a bacterial cell wall derivative such as muramyl dipeptide (MDP) and/or trehalose dicorynomycolate (TDM).

An adjuvant is a substance or procedure which augments specific immune responses to antigens by modulating the activity of immune cells. Examples of adjuvants include, by example only, agonistic antibodies to co-stimulatory molecules, Freunds adjuvant, muramyl dipeptides, liposomes. An adjuvant is therefore an immunomodulator. A carrier is an immunogenic molecule which, when bound to a second molecule augments immune responses to the latter. The term carrier is construed in the following manner. A carrier is an immunogenic molecule which, when bound to a second molecule augments immune responses to the latter. Some antigens are not intrinsically immunogenic yet may be capable of generating antibody responses when associated with a foreign protein molecule such as keyhole-limpet haemocyanin or tetanus toxoid. Such antigens contain B-cell epitopes, but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen.

In a preferred embodiment of the invention said microbial infection is caused by a bacterial species of the genus Clostridium spp.

In a preferred embodiment of the invention said bacterial species is selected from the group consisting of: C. difficile, C. botulinum, C. perfringens or C. tetani.

In a further preferred embodiment of the invention said Clostriduim species is C. difficile.

The vaccine compositions of the invention can be administered by any conventional route, including injection, intranasal spray by inhalation of for example an aerosol or nasal drops. The administration may be, for example, intravenous, intraperitoneal, intramuscular, intracavity, subcutaneous, or intradermally. The vaccine compositions of the invention are administered in effective amounts. An “effective amount” is that amount of a vaccine composition that alone or together with further doses, produces the desired response. In the case of treating a particular bacterial disease the desired response is providing protection when challenged by an infective agent.

The amounts of vaccine will depend, of course, on the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used sufficient to provoke immunity; that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of vaccine administered to a subject can be chosen in accordance with different parameters, in particular in accordance with the mode of administration used and the state of the subject. In the event that a response in a subject is insufficient at the initial doses applied, higher doses (or effectively higher doses by a different, more localized delivery route) may be employed to the extent that patient tolerance permits.

In general, doses of vaccine are formulated and administered in effective immunizing doses according to any standard procedure in the art. Other protocols for the administration of the vaccine compositions will be known to one of ordinary skill in the art, in which the dose amount, schedule of injections, sites of injections, mode of administration and the like vary from the foregoing. Administration of the vaccine compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep or goat.

In a preferred embodiment of the invention there is provided a vaccine composition according to the invention that includes at least one additional anti-bacterial agent.

In a preferred embodiment of the invention said agent is a second different vaccine and/or immunogenic agent (for example a bacterial polypeptide and/or polysaccharide antigen).

According to a further aspect of the invention there is provided a polypeptide as herein described for use in the treatment of microbial infections or conditions that result from microbial infections.

In a preferred embodiment of the invention said microbial infection is a Clostidium infection.

In a preferred embodiment of the invention said condition that results from a microbial infection is selected from the group consisting of: colitis, pseudomembranous colitis, diarrhoea, gangrene, botulism or tetanus.

According to a further aspect of the invention there is provided a method to immunize a subject comprising vaccinating said subject with an effective amount of the polypeptide, nucleic acid molecule or vaccine composition according to the invention.

In a preferred method of the invention said subject is a human.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1a is the nucleotide sequence of processed CD2718; FIG. 1b is the amino acid sequence of mature CD2718.

### MATERIALS AND METHODS

### Strains

Δ630 erm: an erythromycin resistant derivative of the sequenced strain C. difficile strain 630 (Mullany laboratory).

CA434: an E. coli donor strain

The Clostron method of gene inactivation in C. difficile relies on retargeting of a group II intron modified from Lactococcus lactis. In nature this group II intron inserts into ItrB in Lactococcus lactis. This natural system of targeted insertion has been modified by the Minton laboratory to target the group II intron into a gene of interest in Clostridia (Heap et al., 2007).

The target for CD2718 was designed using an algorithm provided by Sigma on the TargeTron website (http://www.sigmaaldrich.com/life-science/functional-genomics-and-rnai/targetron.html). The output from this program provides 3 modified primers IBS, EBS2 and EBS1δ, which are used in a SOE PCR, along with the EBS universal primer and the TargeTron template (Sigma). This SOE PCR incorporates changes (introduced in the 3 modified primers) into the group II intron, which enables the intron to be targeted into the gene of choice. The SOE PCR was performed in accordance with the TargeTron guidelines (Sigma).

The PCR product was then gel extracted using the MinElute Gel extraction kit (Qiagen), and cloned into pGEM T-Easy (Promega) in accordance with the manufacturers' protocol. The insert was then sequenced, after which, restriction digests using HindIII/BsrGI (NEB) were performed in accordance with the manufacturers' protocol. The insert (group II intron) was then ligated into pMTL007, a C. difficile specific plasmid constructed by Heap et al., (2007). The ligation was dialyzed using 0.025 mm white VSWP Filter (Fisher), before being electroporated into One shot TOP10 electro-competent cells (Invitrogen). The insert was then sequenced, before the retargeted pMTL007-CD2718 plasmid was transferred into CA434 electrocompetent E. coli. The retargeting was performed by conjunction with the guidelines provided by the Minton Laboratory². In short, the E. coli donor (strain CA434) carrying pMTL007-CD2718 was mated with stationary phase C. difficileΔ630 erm, by resuspending 1 ml of pelleted E. coli (carrying pMTL007-CD2718) with 200 μl of C. difficile Δ630 erm, under anaerobic conditions. The mating was allowed to occur on non selective BHI plates overnight. The conjugation mixture was resuspended in 1 ml of PBS and plated onto BHI (Brain Heart Infusion) plates containing C. difficile supplement (Fluka), to allow for growth of the C. difficile, but not the E. coli. Colonies were then transferred onto selective plates (BHI+ thiamphenicol) to select for the presence of pMTL007-CD2718 plasmid. The retargeting of the group II intron in the pMTL007-CD2718 was then induced with IPTG, before selection for the presence of the retargeted group II intron in the chromosome, using lincomycin BHI plates (once activated, the group II intron expresses an ermB gene). The loss of pMTL007-CD2817 plasmid was tested using thiamphenicol sensitivity. Clones that were lincomycin resistant and thiamphenicol sensitive were screened by PCR and Southern blot.

### Example

The design and cloning for the sortase knockout was successful and the initial selection for the plasmid was successfully achieved using thiamphenicol (to select for the presence of the pMTL007-CD2718 plasmid). However, the subsequent selection for integration of the intron into the chromosome (Lincomycin selection) and loss of the plasmid were unsuccessful. This was repeated on three different occasions, but no colonies were detected in the lincomycin selection, indicating that the retargeting of the intron had not been successful.

Other targets were successfully mutated alongside targeting the sortase CD2718. For example, two targets in genes involved in the p-cresol production were successfully targeted and mutants were identified. Therefore, construction of gene inactivation mutants in C. difficile strains Δ630 erm and R20291 (a strain from the Stoke Mandeville Hospital outbreak in 2006), were successful. This indicates that the sortase is essential for viability of the organism and therefore mutation was not possible.

### REFERENCES

1 Sebaihia, M. et al. The multidrug-resistant human pathogen Clostridium difficile has a highly mobile, mosaic genome. Nat Genet. 38, 779-786 (2006).
2 Heap, J. T., Pennington, O. J., Cartman, S. T., Carter, G. P. & Minton, N. P. The ClosTron: A universal gene knock-out system for the genus Clostridium. Journal of Microbiological Methods 70, 452-464 (2007).

1. (canceled)

2. A screening method for the identification of an agent that has protease inhibitory activity comprising the steps of:
i) providing a polypeptide encoded by a nucleic acid molecule selected from the group consisting of:
a) a nucleic acid molecule comprising the nucleotide sequence of SEQ ID NO: 1; and
b) a nucleic acid molecule comprising a nucleotide sequence that hybridizes to the sequence identified in (a) under stringent hybridization conditions and which encodes a polypeptide that has protease activity;
ii) providing at least one candidate agent to be tested;
iii) forming a preparation that is a combination of (i) and (ii) above; and
iv) testing the effect of said agent on the activity of said polypeptide.

3. The screening method according to claim 2 wherein said polypeptide comprises or consists of the amino acid sequence of SEQ ID NO: 2, or an active part thereof.

4. (canceled)

5. A polypeptide selected from the group consisting of:
i) a polypeptide encoded by the nucleotide sequence of SEQ ID NO: 1, or an antigenic fragment thereof;
ii) a polypeptide encoded by a nucleotide sequence wherein said sequence is degenerate as a result of the genetic code to the nucleotide sequence defined in (i) and which has protease activity;
iii) a polypeptide comprising an amino acid sequence wherein said sequence is modified by addition deletion or substitution of at least one amino acid residue of SEQ ID NO: 2.

6. The polypeptide according to claim 5 wherein said polypeptide is encoded by the nucleotide sequence of SEQ ID NO: 1.

7. The polypeptide according to claim 5 wherein said polypeptide comprises or consists of the amino acid sequence of SEQ ID NO: 2, or an antigenic part thereof.

8. A nucleic acid molecule that encodes a polypeptide comprising or consisting of the amino acid sequence of SEQ ID NO: 2.

9. A vaccine composition comprising the polypeptide of claim 5 and an adjuvant or carrier.

10. The vaccine composition according to claim 9 wherein said composition includes an adjuvant and a carrier.

11. The vaccine composition according to claim 10 wherein said adjuvant is a cytokine selected from the group consisting of GMCSF, interferon gamma, interferon alpha, interferon beta, interleukin 12, interleukin 23, interleukin 17, interleukin 2, interleukin 1, TGF, TNFα, and TNFβ.

12. The vaccine composition according to claim 10 wherein said adjuvant is a TLR agonist or a bacterial cell wall derivative.

13. The vaccine composition according to claim 12 wherein said bacterial cell wall derivative comprises muramyl dipeptide (MDP) or trehalose dicorynomycolate (TDM).

14. The vaccine composition according to claim 9 further comprising at least one additional anti-bacterial agent.

15. The vaccine composition according to claim 14 wherein said at least one additional anti-bacterial agent is a second different vaccine or immunogenic agent.

16. A method for treating a microbial infection or condition in a subject, comprising administering to the subject an effective amount of the vaccine composition of claim 9.

17. The method according to claim 16 wherein the microbial infection is caused by a bacterial species of the genus Clostridium spp.

18. The method according to claim 17 wherein said bacterial species is selected from the group consisting of: C. difficile, C. botulinum, C. perfringens and C. tetani.

19. The method according to claim 18 wherein said Clostridium species is C. difficile.

20. The method according to claim 16 wherein said condition is selected from the group consisting of: colitis, pseudomembranous colitis, diarrhea, gangrene, botulism and tetanus.

21. The vaccine composition according to claim 12 wherein the TLR agonist comprises CpG oligonucleotides, flagellin, monophosphoryl lipid A, poly I:C or derivatives thereof.
